(19) United States
(12) Patent Application Publication
(10) Pub. No.: US 2016/0274118 A1
(43) Pub. Date: Sep. 22, 2016

(54) BIOMARKER OF LIVER CANCER AND USES THEREOF

(71) Applicant: NATIONAL SUN YAT-SEN UNIVERSITY, Kaohsiung (TW)

(72) Inventors: Ming-Hong Tai, Kaohsiung (TW); San-Cher Chen, Kaohsiung (TW); Tsung-Hui Hu, Kaohsiung (TW); Mei-Lang Kung, Kaohsiung (TW)

(73) Assignee: NATIONAL SUN YAT-SEN UNIVERSITY, Kaohsiung (TW)

(21) Appl. No.: 14/812,269

(22) Filed: Jul. 29, 2015

(30) Foreign Application Priority Data
Mar. 19, 2015 (TW) 104108764

(51) Int. Cl. G01N 33/574

(52) U.S. Cl. CPC G01N 33/57438

(57) ABSTRACT

The present invention relates to uses of nucleolin as a biomarker of liver cancer, comprising predicting, detecting, diagnosing or monitoring liver cancer, high risk of liver cancer, or high risk of vascular invasion of hepatoma cells in a subject, and assessing prognosis of a subject suffered from liver cancer by nucleolin expression level. The biomarkers for determining expression level of nucleolin, kit for determining liver cancer, high risk of liver cancer, or high risk of vascular invasion of hepatoma cells in a subject by expression level of nucleolin, and use of small interfering RNA or antibody specific for recognizing nucleolin in manufacturing medicine for treating liver cancer are also included in the present invention.

### CROSS-REFERENCES TO RELATED APPLICATIONS

The present application claims priority to Taiwan Patent Application No. 104108764 filed on Mar. 19, 2015, incorporated herein by reference in its entirety. The sequence listing text file, file name 2394-NCSU-US_SEQID_LIST created Jul. 29, 2015, file size 8809 bytes, is incorporated herein by reference in its entirety.

### FIELD OF THE INVENTION

The present invention relates to methods of predicting, detecting, diagnosing, monitoring and treating liver cancer. More specifically, the present invention relates to nucleolin-based methods for detecting or treating liver cancer.

### BACKGROUND OF THE INVENTION

Liver cancer or hepatic cancer is a cancer that originates in the liver. The leading cause of liver cancer is cirrhosis due to either hepatitis B, hepatitis C, or alcohol. Primary liver cancer is globally the sixth most frequent cancer, and the second leading cause of cancer death. Many imaging modalities, including sonography (ultrasound), computed tomography (CT) and magnetic resonance imaging (MRI), are used to aid in the diagnosis of primary liver cancer. Tumor markers, chemicals sometimes found in the blood of people with cancer, can be helpful in diagnosing and monitoring the course of liver cancers. However, the causes of liver cancer are quite complicated, so that many new molecular markers of liver cancer have not been discovered.

Screening new molecular markers, with biological characteristics, of liver cancer has been one of the main shafts of liver cancer research. Current clinical serological tests and imaging tests most commonly used in monitoring liver cancer are measuring patient's serum concentration of alpha-fetoprotein (AFP) and liver ultrasound, respectively. The accuracy of detecting liver cancer by serum AFP is only about 25%-60%, therefore, it is considered insufficient as the only way to monitor liver cancer. In addition, tumors less than 2 cm remain a big challenge in diagnosis by pathology and imaging tests. According to the basic medical research in liver cancer, genetic analysis data indicate that the epidermal growth factor receptor (EGFR) is related to liver cancer early recurrence; hepatocyte growth factor (HGF) can be used as a predictor for prognosis and metastasis of liver cancer; transforming growth factor β (TGF-β) over-expression enhances the invasive ability of liver cancer cells; and vascular endothelial growth factor (VEGF) is positively correlated to liver cancer metastasis. However, the actual results still need to be verified. Identifying new molecular markers of liver cancer will be helpful for diagnosing liver cancer, developing treatment programs and determining prognosis.

Early symptoms of liver cancer are not obvious, thus people are easy to miss the gold treatment time. In the past, about 80% of patients diagnosed with liver cancer are already in the case of advanced liver cancer, and may encounter tumor recurrence after surgery. According to statistics, the five-year survival rate of patients with advanced cancer or terminal cancer is less than 25%. Therefore, early detection of liver cancer is very important for extending survival rate of patients. Currently used biomarkers for predicting prognosis or survival rate of liver cancer patients generally can not provide good predictability. In addition, there are still some contradictions for these biomarkers used as diagnostic indicators in the basic medical research stage. Therefore, there remains a need of biomarkers for early diagnosis of liver cancer and methods for accurately predicting the prognosis or overall survival rate of patients with liver cancer. Many basic researches have pointed out that drug resistance arisen during treatment of liver cancer and poor prognosis are relevant to protein expression of oncogenes (MET), vascular endothelial growth factor (VEGF), and hypoxia-inducible factor (HIF-1). There are also researches pointing out that tumor cells in the vicinity of stromal cells trigger over-expression of matrix metalloproteinases (MMP), thus making metastasis of liver tumor and may reduce survival rate. Although the above protein markers are known to be relevant to the growth of liver cancer cells or poor prognosis of liver cancer, however, research results obtained from in vitro experiments of liver cancer cells are not sufficient for being used in clinical diagnosis of liver cancer and prediction of patient's prognosis and survival rate. Therefore, Finding out clear biomarkers with specificity is of great value to early detection and diagnosis of liver cancer.

Nucleolin is a major protein in nucleoli of eukaryotic nuclei, which has extensive functionality, such as regulating synthesis and maturation of ribosomes, stability of nucleolar chromatin organization, cell proliferation and growth, embryogenesis, cytokinesis and cell stress response, and anti-apoptotic effects. When tumor cells and vascular endothelial cells proliferate, nucleolin can act as a multifunctional shuttle protein which shuttles between the cytoplasm and the nucleus. Moreover, nucleolin can express on the cell membrane to serve as a variety of protein molecules such as: endothelial growth factor, lipoproteins, endostatin and certain viral receptors. In cervical cancer, melanoma and breast cancer cells, nucleolin is excessively expressed. In addition, nucleolin over-expression in nuclei is positively correlated to tumor progression of children intracranial ependymoma and melanotic cancer. However, in the clinical analysis of pancreatic ductal adenocarcinoma, it is found that if nucleolin in a patient's cancer cells is over-expressed, then the patient has a higher overall survival rate. Also, it is found that neither the degree of malignancy nor the tumor size of pancreatic ductal adenocarcinoma is correlated with over-expression of nucleolin (Clin Cancer Res. 2010 Jul. 15; 16(14):3734-42). So far, there is neither publication nor patent reporting the relevance between nucleolin expression and diagnosis of liver cancer or prognosis of liver cancer patients after surgery.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows liver cancer cells SK-Hep1 with immunofluorescence staining to analyze the distribution of nucleolin protein. (A): Triton X-100 solution with concentration of 2% is used to create holes on the cell surface, so that the anti-nucleolin antibody can enter into cells and be stained as fluorescent green. (B): Triton X-100 solution is not used to maintain integrity of cell membrane, and the anti-nucleolin antibody is stained on the cell surface as fluorescent green. (C) and (D): The fluorescent dye 4′,6-diamidino-2-phenylindole (DAPI) is used to color nuclei, and the part of fluorescent blue represents the position of nucleus. Scale bar: 20 μm.

FIG. 2 shows western blot analysis results of different liver cancer cell lines for analyzing expression level of nucleolin protein. Liver cancer cell lines arranged from left to right indicate from low to high malignancy of cancer cells. (A): Expression level of nucleolin protein is analyzed by total protein mass of different liver cancer cell lines, and β-actin is used as an internal control. (B): A histogram which indicates expression level of nucleolin protein from different liver cancer cell lines by western blot analysis results, and nucleolin protein level is represented by multiples/β-actin expression amount (β-actin level is used as an internal control). (C): Expression level of nucleolin protein is analyzed by membrane protein mass of different liver cancer cell lines, and Pan-cadherin is used as an internal control. (D): A histogram which indicates expression level of nucleolin protein from different liver cancer cell lines by western blot analysis results, and nucleolin protein level is represented by multiples/Pan-cadherin expression amount (Pan-cadherin level is used as an internal control). Statistical analysis is carried out by using SPSS v17 software to analyze the data. Student's t-test is used to quantitate variables. The two-sided p value less than 0.05 (<0.05) is regarded as significant. The expressed experimental data is the average of three independent experiments.

FIG. 3 shows cell proliferation phenomenon caused by transfection of GFP-nucleolin expression plasmid into liver cancer cells (SK-Hep1). By cell viability assay, the absorbance is measured and presented in the figure. The higher the absorbance value represents the higher cell proliferation.

FIG. 4 shows results of immunohistochemical staining and immunofluorescence staining for analyzing differences between nucleolin protein expression level in rat liver tumor and nucleolin protein expression level in non-tumor tissue surrounding the liver tumor. (A): Immunohistochemical staining showing nucleolin protein expression level in liver tumor tissue (T) and surrounding non-tumor tissue (N) of rats (100× magnification). (B): Nucleolin protein expression level in liver non-tumor tissue (N) of rats is presented by capturing and enlarging N region in (A) (400× magnification). (C): Nucleolin protein expression level in liver tumor tissue (T) of rats is presented by capturing and enlarging T region in (A) (400× magnification). (D): Immunofluorescence staining showing nucleolin protein expression level in liver non-tumor tissue (N) of rats (200× magnification). (E): Immunofluorescence staining showing nucleolin protein expression level in liver tumor tissue (T) of rats (200× magnification).

FIG. 5 shows results of immunohistochemical staining for comparing differences between nucleolin protein expression level in human liver tumor and nucleolin protein expression level in non-tumor tissue surrounding the liver tumor. (A): Immunohistochemical staining showing nucleolin protein expression level in liver tumor tissue (T) and surrounding non-tumor tissue (N) of humans (100× magnification). (B): Nucleolin protein expression level in liver non-tumor tissue (N) of humans is presented by capturing and enlarging N region in (A) (400× magnification). (C): Nucleolin protein expression level in liver tumor tissue (T) of humans is presented by capturing and enlarging T region in (A) (400× magnification). Statistical analysis is carried out by using SPSS v17 software to analyze the data. Student's t-test is used to quantitate variables. The two-sided p value less than 0.05 (<0.05) is regarded as significant.

FIG. 6 shows the Kaplan—Meier plot for representing the relationship between nucleolin expression level in human liver tissue samples and overall survival of patients with liver cancer.

FIG. 7 shows effects of transfecting interfering nucleolin RNA on cell growth and invasive ability of human liver cancer cell line Huh7. (A): The cell proliferation of Huh7 cells, Huh7 cells interfered by transfection of scrambled RNA, and Huh7 cells interfered by transfection of nucleolin RNA is analyzed by cell viability assay. (B): The cell invasion of Huh7 cells, Huh7 cells interfered by transfection of scrambled RNA, and Huh7 cells interfered by transfection of nucleolin RNA is analyzed by cell invasion assay. The upper photos are representative of cell invasion ability. Statistical analysis is carried out by using SPSS v17 software to analyze the data. Student's t-test is used to quantitate variables. The two-sided p value less than 0.05 (<0.05) is regarded as significant. The expressed experimental data is the average of three independent experiments.

FIG. 8 shows effects of nucleolin antibody neutralization on cell growth and invasive ability of human liver cancer cell line Huh7. (A): The cell proliferation of Huh7 cells, Huh7 cells neutralized by immunoglobulin G antibody, and Huh7 cells neutralized by nucleolin antibody is analyzed by cell viability assay. (B): The cell invasion of Huh7 cells, Huh7 cells neutralized by immunoglobulin G antibody, and Huh7 cells neutralized by nucleolin antibody is analyzed by cell invasion assay. The upper photos are representative of cell invasion ability. Statistical analysis is carried out by using SPSS v17 software to analyze the data. Student's t-test is used to quantitate variables. The two-sided p value less than 0.05 (<0.05) is regarded as significant. The expressed experimental data is the average of three independent experiments.

### DETAILED DESCRIPTION OF THE INVENTION

In order to overcome the shortcomings of the above-mentioned conventional technology, the present invention provides a biomarker of liver cancer and a detection method thereof.

The liver cancer biomarker of the present invention is necleolin. In vitro experimental model of liver cancer cells and analysis of ex vivo liver tissue samples after surgery show that nucleolin expression level is positively correlated with malignancy and vascular invasion of liver cancer cells. Over-expression of nucleolin in liver cancer cells will promote the growth of cancer cells. The higher the nucleolin expression level in liver cancer tissues, the worse the prognosis and survival rate of patients are. In contrast, inhibition of nucleolin expression in liver cancer cells can reduce the growth and invasion ability of cancer cells. Therefore, it is believed that the liver cancer biomarker nucleolin and the detection methods thereof can provide a method available to detect or diagnose high-risk of liver cancer or prognosis and survival rate of patients.

The main object of the present invention to provide a liver cancer biomarker nucleolin and detection methods thereof, which are used to assist in diagnosing if the liver cancer is high risk, distinguish malignant degree of cancer cells, or distinguish vascular invasion degree of cancer cells, thus help understand liver cancer patient's condition, thereby prescribe the right medicine and improve patient outcomes. The liver cancer biomarker nucleolin and detection methods thereof provided by the present invention may combine with existing examination methods, for example, determine alpha-fetoprotein in serem, ultrasound examination, CT scan (computer tomography) or MRI (magnetic resonance imaging) in order to facilitate the detection of liver cancer or determination of prognosis or survival rate of patients.

Therefore, the present invention provides a method for determining liver cancer, high risk of liver cancer, or high risk of vascular invasion of liver cancer cells in a subject, comprising steps of:
(A) assessing nucleolin expression level in a biological sample obtained from the subject;
(B) comparing the nucleolin expression level with that in a corresponding control sample; and
(C) determining whether the subject has liver cancer, high risk of liver cancer, or high risk of vascular invasion of liver cancer cells in accordance with the result of step (B); wherein the nucleolin expression level in the biological sample that is higher than that in the corresponding control sample is indicative of the subject having liver cancer, high risk of liver cancer, or high risk of vascular invasion of liver cancer cells.

The nucleolin expression level may be assessed by the following assays, including, but not limited to, immunofluorescence staining, western blot, or immunohistochemical staining. The biological sample may be a liver cell sample, a biopsy sample of liver tissue, a liver homogenate sample, or any combination thereof. The corresponding control sample is obtained from a healthy subject, and the healthy subject refers to a subject not suffering from liver cancer. The above method may combine with an examination method selected from the group consisting of serem alpha-fetoprotein, ultrasound examination, CT scan (computer tomography) and MRI (magnetic resonance imaging) in order to facilitate detection of liver cancer and/or determination of cancer tissue invasion or malignancy or vascular invasion.

The present invention also provides a biomarker for determining liver cancer, high risk of liver cancer, or high risk of vascular invasion of liver cancer cells in a subject, comprising a biomarker for determining nucleolin expression level, wherein the nucleolin expression level that is higher than that in the corresponding control sample is indicative of the subject having liver cancer, high risk of liver cancer, or high risk of vascular invasion of liver cancer cells. The biomarker for determining nucleolin expression level may be, for example (including but not limited to): hepatoma-derived growth factor (HDGF), phosphatidylinositol 3-kinase (PI3K), protein kinase B (AKT), or mammalian target of rapamycin (mTOR).

The present invention further provides a kit for determining liver cancer, high risk of liver cancer, or high risk of vascular invasion of liver cancer cells in a subject, comprising: a biomarker detection reagent for assessing nucleolin expression level in a biological sample, and a manual regarding using the biomarker detection reagent for determining liver cancer, high risk of liver cancer, or high risk of vascular invasion of liver cancer cells, wherein the manual comprises instructions for comparing the nucleolin expression level in the biological sample with that in a corresponding control sample.

The present invention still further provides a method for estimating prognosis in a subject having liver cancer, comprising: providing a biological sample obtained from the subject; assessing nucleolin expression level in the biological sample; and comparing the nucleolin expression level with that in a corresponding control sample; wherein the nucleolin expression level that is higher than that in the corresponding control sample is indicative of the subject having poor prognosis or overall survival rate.

The present invention yet still further provides use of an agent for preparing a medicament for treating liver cancer, wherein the agent comprises a small interfering RNA of nucleolin or an antibody which specifically recognizes nucleolin. The present invention yet still further provides a method for treating liver cancer in a patient in need thereof comprising: administering to said patient a pharmaceutically effective amount of composition comprising a small interfering RNA of nucleolin or an antibody which specifically recognizes nucleolin. The antibody which specifically recognizes nucleolin may be a monoclonal antibody, a polyclonal antibody, or a single chain antibody. In an embodiment, the phrase “treating liver cancer” refers to inhibiting or treating proliferation or invasion of liver cancer cells.

When the method of the present invention is practiced in vitro, the liver cancer cells may derive from liver cancer cell lines. Examples of appropriate liver cancer cell lines include, but not limited to, HepG2, Hep3B, Mahlavu, J5, Huh-7 and SK-Hep-1 cell lines.

The term “subject” used in the present invention includes, but not limited to, a mammalian subject. Preferably, the mammalian subject is a human or rodent subject.

The method for determining cancer provided by the present invention has the following advantages when being compared with the aforementioned conventional technology:
(1) The cancer detection method provided by the present invention uses nucleolin expression level in the tested sample as a diagnostic indicator for cancer or high risk of cancer or vascular invasion of cancer cells. This method can be used for first-line screening of liver cancer, or combined with alpha-fetoprotein test and liver ultrasound for second-line screening of liver cancer to facilitate more accurate diagnosis and treatment of liver cancer.
(2) The cancer detection method provided by the present invention not only can be applied in detection of liver cancer, but also can be applied in detection of other cancers such as breast cancer, stomach cancer, colon cancer to assist diagnosis of abnormal samples.

For a more complete understanding of the features and advantages of the present invention, the examples of the present invention and the accompanying drawings are for reference. Although the implementation and usage of each example of the present invention are discussed in detail below, it should be understood that the present invention provides many applicable inventive concepts which can be implemented by various specific content. Specific embodiments described herein do not define the scope of the invention but are merely illustrative of specific methods for implementing and using the present invention.

Unless otherwise defined in the present context, the scientific and technical terms used in the present invention should possess meaning commonly known by any person with ordinary skill in the art. The meaning and scope of the terms should be clear; nevertheless, in any circumstance of discrepancy in the meaning, definition provided in the present context precedes those defined in any other dictionaries or external references.

The entire context of any references cited in the present context is incorporated into the present context as references.

Unless otherwise needed in the present context, singular terms should include plural forms and plural terms should include singular forms.

As used herein, the term “nucleolin” refers to a protein in nucleoli of eukaryotic nuclei, which can regulate synthesis and maturation of ribosomes, stability of nucleolar chromatin organization, cell proliferation and growth, embryogenesis, cytokinesis and cell stress response, and also has anti-apoptotic effects. In the present invention, examples of the amino acid sequence of the nucleolin include but not limited to SEQ ID NO: 1.

As used herein, the term “liver cancer” includes medical definition that can be widely accepted, which define liver cancer as a medical condition having cancer characteristics in which liver cells mutate and constantly divide, thus undermine normal liver tissue, even transfer to other body organs.

As used herein, the terms “sample”, “tissue sample”, “tissue biopsy sample”, “cell sample”, and “cell homogenate sample” all refer to similar collection of cells obtained from patient's tissue. The source of the tissue sample may be fresh, frozen and/or stored organ or tissue sample, blood, or any blood component.

As used herein, the term “normal sample”, “normal tissue”, or “control sample” refers to disease-free or normal cells or tissues for being compared with measured value of samples obtained from patients to determine whether the nucleolin protein amount is small in the normal sample, or whether the nucleolin protein amount is excess in the sample obtained from patients. The normal sample may be obtained from normal tissues adjacent to tumors in patients themselves, or obtained from healthy individuals. Preferably, the nucleolin protein amount either in the normal sample or in the corresponding patient sample is obtained under the same experimental condition. The normal sample may be obtained from the same tissue as the patient sample. In the assay of the present invention, the nucleolin protein amounts in normal samples are tabulated in advance to provide an average range, mean value and standard deviation, or similar expressions, then the nucleolin protein amounts in patient samples are compared with corresponding values in the normal samples.

As used herein, the term “gene” refers to a functional protein-, polypeptide- or peptide-encoding unit. As will be understood by those in the art, this functional term includes both genomic sequences, cDNA sequences and smaller engineered gene segments that express, or may be adapted to express, proteins, polypeptides or peptides.

As used herein, the term “biomarker” refers to a specific protein molecule which can be used for diagnosing and measuring disease progression or for treatment in an organism. The main types of cancer biomarkers based on clinical utility and application include the following: (1) “Diagnostic biomarkers” for determining whether patients suffer from cancer or defining the type of cancer. The diagnostic biomarkers can also be used for detecting or defining recurrent disease after first-line treatment. (2) “Prognostic biomarkers” for indicating status or possibility of cancer metastasis and/or possible tumor's growth rate, which estimate the patients' outcome regardless of the type of treatment. (3) “Predictive biomarkers” which are used to identify subpopulations of patients who are most likely to respond to a given therapy. (4) “Medicine dynamic biomarkers” or “pharmacological biomarkers” which can help identify a drug dose for an individual. (5) Biomarkers used for monitoring patient response to treatment, so that the treatment regimen (drug or dose) can be modified if necessary. The biomarkers of the present invention can be used as the above-mentioned five types for all relevant purposes.

As used herein, the term “suppress”, “decrease” or “reduce” or any variation of these terms when used in the claims and/or specification includes any measurable reduction or complete inhibition to achieve the desired result.

As used herein, the term “prognosis” refers to the prediction of possible progression and outcome of disease, which includes determining the specific consequences of the disease, such as rehabilitation, appearance or disappearance of certain symptoms, signs, complications, or other abnormalities, and death.

As used herein, the term “surgery” refers to resection of liver tumors by surgical operation. The range of resection is based on the location of the tumor occurred, and the size and visually observed pattern of the tumor, in order to reach the purpose of no residual cancer cells.

As used herein, the term “immunohistochemistry” (also called “immunofluorescence staining” when applied in cells) refers to a dyeing method in diagnostic pathology, in which a given protein antibody is used for the diagnosis of the difference of the given protein expression amount in tumors.

As used herein, the term “small interfering RNA” or “siRNA”, also called “short interfering RNA” or “silencing RNA” refers to a double-stranded RNA with 20-25 nucleotides in length, which mainly involves in RNA interference (RNAi) by regulating gene expression specifically. In addition, siRNA also involves in some reaction pathways relevant to RNAi, such as antiviral mechanism or changes of chromatin structure. siRNA is typically a 21-bp double-stranded RNA (dsRNA) with phosphorylated 5′ ends and hydroxylated 3′ ends with two overhanging nucleotides (such as tt, t means deoxythymidine (dT)). The Dicer enzyme catalyzes production of siRNAs from long dsRNAs and small hairpin RNAs. siRNAs can also be introduced into cells by transfection. Since in principle any gene can be knocked down by a synthetic siRNA with a complementary sequence, siRNAs are an important tool for validating gene function and drug targeting in the post-genomic era. Each siRNA is unwound into two single-stranded RNAs (ssRNAs), the passenger strand and the guide strand. The passenger strand is degraded and the guide strand is incorporated into the RNA-induced silencing complex (RISC). The guide strand acts as a template for RISC to recognize complementary messenger RNA (mRNA) transcript. Once found, one of the proteins in RISC, called Argonaute, activates and cleaves the mRNA. This process is called RNA interference and it is found in many eukaryotes; it is a key process in gene silencing and defense against viral infections.

When the term “one” is used in conjunction with the term “comprising” in claims and/or specification, it means “one”, but also equivalent to the meaning of “one or more”, “at least one” and “one or more than one”.

The term “or” used in the claims refers to “and/or” unless substitutes are mutually exclusive.

The term “about” in the full text of the present application refers to a value comprising inherent error differences derived from the methods or devices used for determining the value, or differences exist in the subjects.

As used in the specification and claims, the term “comprise” (and any form of comprise), “have” (and any form of have), “include” (and any form of include), or “contain” (and any form of contain) indicates inclusion rather than limitation. When a patent claim states that a particular product or method comprises certain elements, this means that other elements may also be present.

As used herein, the term “or the combination thereof” refers to all permutations and combinations of the items listed before this term. For example, “A, B, C, or the combination thereof” includes at least one of the following: A, B, C, AB, AC, BC, and ABC. If it is order-related in particular content, then BA, CA, CB, CBA, BCA, ACB, BAC, or CAB is also included. In this example, combination containing duplicates of items is also included, such as BB, AAA, MB, BBC, AAABCCCC, CBBAAA, or CABABB. The skilled in the art will appreciate that, unless explicitly stated, limitations on the number of combinations of items and terms generally do not exist.

The term “statistically significant” or “statistical significance” refers to the likelihood that a result would have occurred by chance, given that an independent variable has no effect, or, that a presumed null hypothesis is true. Statistical significance can be determined by obtaining a “P-value” (P) which refers to the probability value. The p-value indicates how likely it is that the result obtained by the experiment is due to chance alone. In one embodiment of the invention, statistical significance can be determined by obtaining the p-value of chi-square test or student's t-test. A p-value of less than 0.05 is considered statistically significant, that is, not likely to be due to chance alone.

### EXAMPLES

The examples below are non-limiting and are merely representative of various aspects and features of the present invention.

### Example 1

### Distribution of Nucleolin Protein in Liver Cells

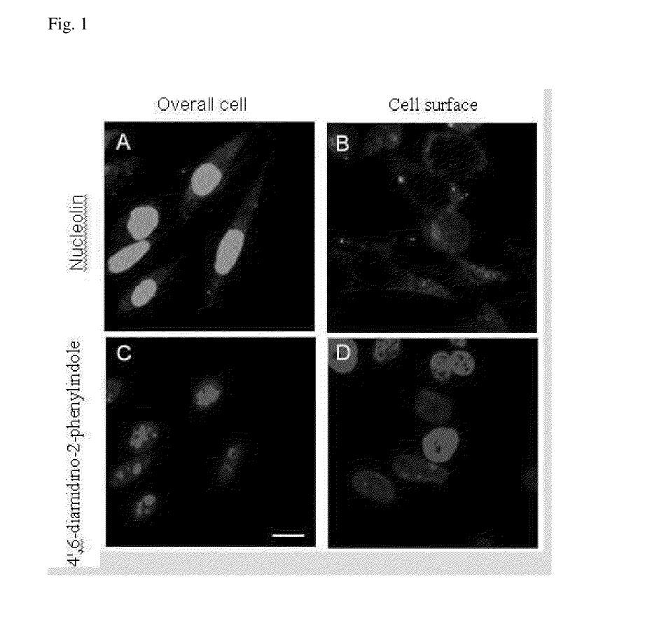

Liver cancer cells SK-Hep1 were washed by phosphate buffer solution three times, and then 4% paraformadehyde was added as a fixative. All cells were covered by the fixative and allowed to stand at 37° C. for 10 minutes. The fixative was removed, followed by addition of phosphate buffer solution. After slowly shaken at room temperature for 1 hour, the cells were washed by phosphate buffer solution three times. The cells to be holed were soaked in Triton X-100 solution with concentration of 2% for 10 minutes, washed by phosphate buffer solution three times, and then 1% bovine serum albumin was used to remove non-specific binding. After slowly shaken at room temperature for 1 hour to absorb 1% bovine serum albumin, the cells were washed by phosphate buffer solution three times. Nucleolin primary antibody (purchased from Santa Cruz Biotechnology, Santa Cruz, Calif.; Catalog No.: sc-8031) diluted 200 times was added, slowly shaken at 4° C. overnight, and then washed by phosphate buffer solution five times (5 minutes each time). After washing was completed, secondary antibody (purchased from Santa Cruz Biotechnology, Santa Cruz, Calif.; Catalog No.: sc-2005) diluted 800 times was added for reaction at room temperature for 1 hour, and then washed by phosphate buffer solution five times at room temperature (5 minutes each time). After washing was completed, the solution containing nuclear fluorescent dye 4′,6-diamidino-2-phenylindole (DAPI) diluted 10000 times was added for reaction at room temperature for 5 minutes, and then washed by phosphate buffer solution five times at room temperature (5 minutes each time). After washing was completed, coverslips were air dried with cell side upward. A drop of mounting solution was dropped onto the slide and the coverslip was placed cell side down onto the drop of mounting solution to complete slide mounting. Photographs were taken by a fluorescence microscope and the results were shown in FIG. 1.

FIG. 1 showed liver cancer cells SK-Hep1 with immunofluorescence staining to analyze the distribution of nucleolin protein. (A): Triton X-100 solution with concentration of 2% was used to create holes on the cell surface, so that the anti-nucleolin antibody could enter into cells and stain as fluorescent green. (B): Triton X-100 solution was not used to maintain integrity of cell membrane, so that the anti-nucleolin antibody stained on the cell surface as fluorescent green. (C) and (D): The nuclear fluorescent dye 4′,6-diamidino-2-phenylindole (DAPI) entered into cells and colored nuclei, and the part of fluorescent blue represented the position of nucleus. Scale bar: 20 μm.

According to (A) and (C) of FIG. 1, the results showed that nucleolin distributed throughout the liver cancer cell. Nucleolin protein was expressed mainly in the nucleus, but its expression was also observed in the cytoplasm and cell membrane. From (B) and (D) of FIG. 1, the results showed that when the cell membrane was maintained integral, the anti-nucleolin antibody bound to the surface nucleolin protein on the cell surface, indicating that nucleolin protein expression could be clearly observed in the cell membranes of cancer liver cells.

### Example 2

### Nucleolin Protein Expression Level in Different Liver Cancer Cell Lines

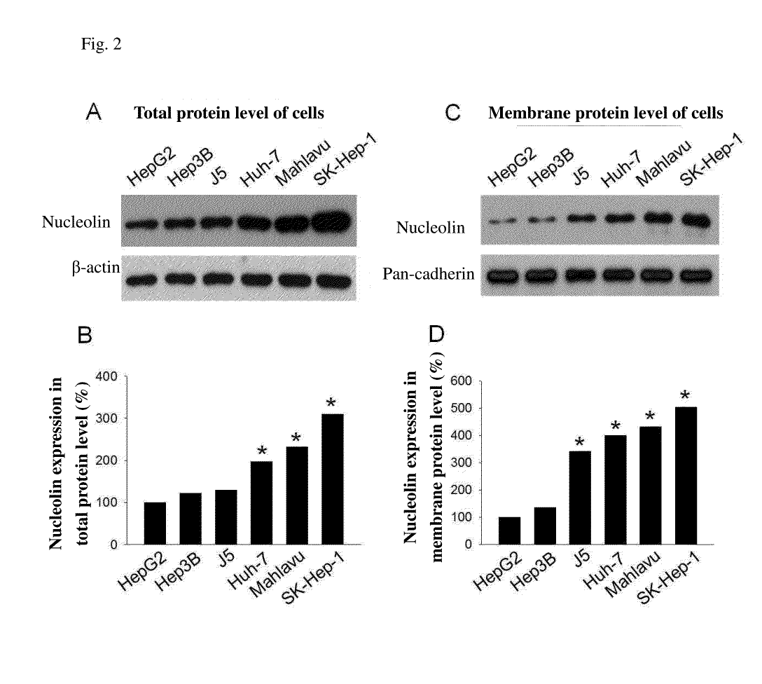

The human liver cancer cell lines HepG2 and Hep3B used in the example were cultured in MEM medium supplemented with 10% fetal bovine serum (FBS, purchased from Invitrogen Carlsbad, USA). Mahlavu, J5, Huh-7, and SK-Hep-1 cells were cultured in DMEM medium supplemented with 10% FBS. All mediums were additionally added 100 U/ml penicillin and 100 μg/ml streptomycin and were incubated at 37° C. with carbon dioxide concentration of 5%, and subcultured every 2-3 days. The cell lysis buffer was added in liver cancer cells, and the cellular protein was extracted. The protein solution was quantitated, and then ⅕ of the total amount of dye was added. The resultant mixture was cooked at 100° C. for 5 minutes, and centrifuged at 4° C. The cooked sample was added into 10% SDS-PAGE gel wells. The electrophoresis was carried out by running the protein through the stacking gel at 80 volts and through the resolving gel at 100 volts. The protein on the gel was transferred onto PVDF (Polyvinylidene fluoride) which was pre-treated by being wet in methanol. The transfer was carried out at 100 mA for 2 hours. After transfer, the PVDF was blocked by being soaked in the blocking buffer containing 5% fat free milk and TBST for 1 hour. The PVDF was then washed by TBST four times, 5 minutes each time. The PVDF was then soaked in the phosphate buffer solution containing nucleolin, β-actin or Pan-cadherin primary antibody (all of them were purchased from Santa Cruz Biotechnology, Santa Cruz, Calif.; Catalog No.: sc-8031, sc-47778, and sc-59876, respectively), and slowly shaken at 4° C. overnight for reaction, and then washed by phosphate buffer solution four times (5 minutes each time). The PVDF was then soaked in the phosphate buffer solution containing horseradish peroxidase-linked secondary antibody (purchased from Santa Cruz Biotechnology, Santa Cruz, Calif.; Catalog No.: sc-2005) at room temperature for 1 hour of reaction, and then washed by phosphate buffer solution four times (5 minutes each time). Finally, an enhanced chemiluminescence detection reagent was used for detection, and the images were shown on X-ray films. The results were shown in FIG. 2.

FIG. 2 showed western blot analysis results of different liver cancer cell lines for analyzing expression level of nucleolin protein. Liver cancer cell lines arranged from left to right indicated from low to high malignancy of cancer cells. (A): Expression level of nucleolin protein was analyzed by total protein mass of different liver cancer cell lines, and β-actin was used as an internal control. (B): A histogram which indicated expression level of nucleolin protein from different liver cancer cell lines by western blot analysis results, and nucleolin protein level was represented by multiples/β-actin expression amount (β-actin level was used as an internal control). (C): Expression level of nucleolin protein was analyzed by membrane protein mass of different liver cancer cell lines, and Pan-cadherin was used as an internal control. (D): A histogram which indicated expression level of nucleolin protein from different liver cancer cell lines by western blot analysis results, and nucleolin protein level was represented by multiples/Pan-cadherin expression amount (Pan-cadherin level was used as an internal control). Statistical analysis was carried out by using SPSS v17 software to analyze the data. Student's t-test was used to quantitate variables. The two-sided p value less than 0.05 (<0.05) is regarded as significant. The expressed experimental data is the average of three independent experiments.

From (A) and (B) of FIG. 2, the results showed that nucleolin protein expression was observed in cell homogenate samples of liver cancer cells with different malignancy, comprising low malignant liver cancer cells (HepG2, Hep3B) and high malignant liver cancer cells (Mahlavu, SK-Hep-1). The nucleolin protein expression level was found being positively correlated with malignancy of liver cancer cells. From (C) and (D) of FIG. 2, the results showed that nucleolin protein expression was also observed in membrane homogenate samples of liver cancer cells, and the nucleolin protein expression level was also positively correlated with malignancy of liver cancer cells. The results of (B) and (D) in FIG. 2 were generated by quantitating electrophoresis results of (A) and (C) respectively followed by statistics.

### Example 3

### Effect of Nucleolin on Liver Cancer Cell Proliferation

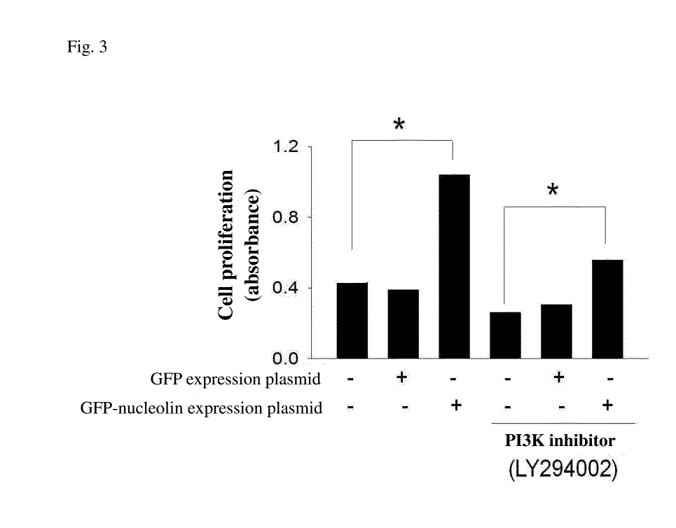

cDNAs obtained from reverse transcription of RNA extracted from liver cancer cells (SK-Hep-1 cell line) were subjected to amplification of nucleolin by using forward primer (5′-atggtgaagctcgcgaaggcag-3′, SEQ ID NO: 2) and reverse primer (5′-ttcaaacttcgtcttctttccttg-3′, SEQ ID NO: 3) for nucleolin, and reverse transcription polymerase. The amplification product was inserted into green fluorescent protein (GFP) expression plasmid to form GFP-nucleolin expression plasmid. This GFP-nucleolin expression plasmid was transiently transfected into human liver cancer cells, so that the cells could express a lot of nucleolin protein. 6×10⁵ liver cancer cells were cultured in a 6-well sterile petri dish for 24 hours at 37° C. Then GFP-nucleolin expression plasmid and a control group of GFP expression plasmid were transfected into liver cancer cells by Opti-MEM transfection reagent (purchased from Gibco) in accordance with the reagent instruction, followed by cell proliferation experiment. MTT assay was an analysis method common for analyzing cell proliferation or survival. MTT (3-[4,5-dimethylthiazol-2-yl]2,5-diphenyltetrazolium bromide) was a yellow dye, which could be absorbed by living cells and reduced to water-insoluble blue-purple formazan by succinate tetrazolium reductase in mitochondria. Therefore, cell proliferation could be determined and analyzed by formation of formazan. 5×10⁴ cells were cultured in a 24-well plate for 24 hours, and GFP-nucleolin expression plasmid and a control group of GFP expression plasmid were transfected to the cells for 48 hours. 100 μg of MTT reagent was added per well during culture. After placed at 37° C. for 4 hours, 500 μl of dimethyl sulfoxide was added per well to dissolve the formazan precipitate formed. The plate was left to stand for 10 minutes, and then enzyme-linked immunosorbent assay (ELISA) was carried out and the absorbance was read. The results were shown in FIG. 3.

FIG. 3 showed cell proliferation phenomenon caused by transfection of GFP-nucleolin expression plasmid into liver cancer cells (SK-Hep1). By cell viability assay, the absorbance is measured and presented in the figure. The higher the absorbance value represents the higher cell proliferation. GFP group (control): SK-Hep1 cells transfected with GFP expression plasmid which had no effect on cell growth. GFP-nucleolin group: SK-Hep1 cells transfected with GFP-nucleolin expression plasmid. LY294002: An artificially synthesized PI3K inhibitor which inhibited cell growth. “+”: with treatment; “−”: without treatment. Statistical analysis is carried out by using SPSS v17 software to analyze the data. Student's t-test is used to quantitate variables. The two-sided p value less than 0.05 (<0.05) is regarded as significant. The expressed experimental data is the average of three independent experiments.

According to FIG. 3, SK-Hep1 cells transfected with GFP-nucleolin expression plasmid was about 2.5 times the cell proliferation rate of the control group. Even when the cell growth inhibitor LY294002 inhibited cell proliferation, liver cancer cells transfected with GFP-nucleolin expression plasmid could still promote cell proliferation.

### Example 4

### Difference Between Nucleolin Protein Expression Level in Rat Liver Tumor and that in Non-Tumor Tissue Surrounding the Liver Tumor

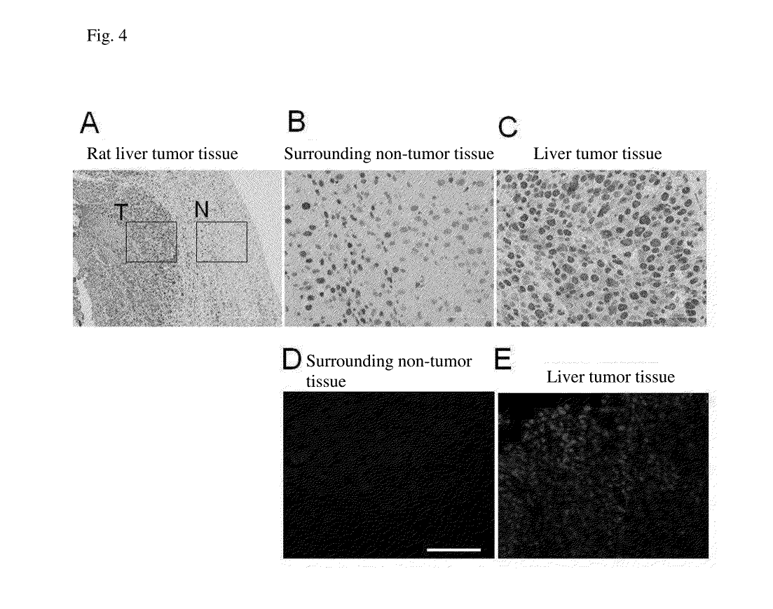

The tissue samples of rats were embedded in paraffin. The samples were sliced by a microtome and the slices were affixed to glass slides. The paraffin-embedded tissue samples on the glass slides were rehydrated before 3% hydrogen peroxide was added to block activity of endogenous peroxidase. The samples were placed in horse serum diluted 20-fold at room temperature for 30 minutes of blocking reaction. For immunohistochemistry, nucleolin primary antibody (purchased from Santa Cruz Biotechnology, Santa Cruz, Calif.; Catalog No.: sc-8031) diluted 100 times was added into the samples and slowly shaken at 4° C. overnight. After the samples were washed by phosphate buffer solution three times, secondary antibody (purchased from Santa Cruz Biotechnology, Santa Cruz, Calif.; Catalog No.: sc-2005) diluted 1000 times was added for reaction at room temperature for 30 minutes, and then washed by phosphate buffer solution three times at room temperature (5 minutes each time). After washing was completed, diaminobenzidine (DAB) was used for staining. After washing again, the samples were counterstained with hematoxylin, followed by dehydration with graded ethanol, and fixation with xylene. The experimental results were photoed with an optical microscope for analysis. For immunofluorescence staining, nucleolin primary antibody (purchased from Santa Cruz Biotechnology, Santa Cruz, Calif.; Catalog No.: sc-8031) diluted 200 times was added into the samples and slowly shaken at 4° C. overnight. After the samples were washed by phosphate buffer solution five times (5 minutes each time), secondary antibody (purchased from Santa Cruz Biotechnology, Santa Cruz, Calif.; Catalog No.: sc-2005) diluted 1000 times was added for reaction at room temperature for 1 hour, and then washed by phosphate buffer solution five times at room temperature (5 minutes each time). After washing was completed, the solution containing nuclear fluorescent dye 4′,6-diamidino-2-phenylindole (DAPI) diluted 10000 times was added for reaction at room temperature for 5 minutes, and then washed by phosphate buffer solution five times at room temperature (5 minutes each time). After washing was completed, coverslips were air dried with cell side upward. A drop of mounting solution was dropped onto the slide and the coverslip was placed cell side down onto the drop of mounting solution to complete slide mounting. Photographs were taken by a fluorescence microscope and the results were shown in FIG. 4. Interpretation of the results obtained from the immunohistochemical staining of nucleolin was done by two pathologists independently. Four levels were defined according to the immune response of nucleolin in the nucleus: 0 represented no nuclear staining; 1⁺ represented 1-25% nuclear staining; 2⁺ represented 26-50% nuclear staining; 3⁺ represented >50% nuclear staining. Only 3⁺ was identified as a positive staining result.

FIG. 4 showed results of immunohistochemical staining and immunofluorescence staining for analyzing differences between nucleolin protein expression level in rat liver tumor and that in non-tumor tissue surrounding the liver tumor. (A): Immunohistochemical staining showing nucleolin protein expression level in liver tumor tissue (T) and surrounding non-tumor tissue (N) of rats (100× magnification). (B): Nucleolin protein expression level in liver non-tumor tissue (N) of rats was presented by capturing and enlarging N region in (A) (400× magnification). (C): Nucleolin protein expression level in liver tumor tissue (T) of rats was presented by capturing and enlarging T region in (A) (400× magnification). (D): Immunofluorescence staining showing nucleolin protein expression level in liver non-tumor tissue (N) of rats (200× magnification). (E): Immunofluorescence staining showing nucleolin protein expression level in liver tumor tissue (T) of rats (200× magnification).

From (A) of FIG. 4, the immunohistochemical staining result showed that nucleolin protein expression level in rat liver tumor was significantly higher than that in non-tumor tissue surrounding the liver tumor. According to (B) and (C) of FIG. 4, nucleolin showed positive staining in the nucleus, cytoplasm and cell membrane of the cancer cell in tumor tissue (determined by tumor tissue staining results greater than or equal to 50% of the nuclear staining). Traces of nucleolin were found in the non-tumor tissue. Nucleolin expression amount in the liver tumor tissue was significantly higher than that in the non-tumor tissue. According to (D) and (E) of FIG. 4, it was reconfirmed by immunofluorescence staining that nucleolin expression amount in the liver tumor tissue was significantly higher than that in the non-tumor tissue.

### Example 5

### Difference Between Nucleolin Protein Expression Level in Human Liver Tumor and that in Non-Tumor Tissue Surrounding the Liver Tumor

A retrospective analysis of the liver cancer patients which had been treated in Kaohsiung Chang Gung Memorial Hospital from January 1987 to December 1998 was undertaken. This study was approved by Institutional Review Board (IRB) of Kaohsiung Chang Gung Memorial Hospital. Primary tumor samples and corresponding non-tumor tissue were obtained in surgery. The tissue samples of patients were embedded in paraffin. The samples were sliced by a microtome and the slices were affixed to glass slides. The paraffin-embedded tissue samples on the glass slides were rehydrated before 3% hydrogen peroxide was added to block activity of endogenous peroxidase. The samples were placed in horse serum diluted 20-fold at room temperature for 30 minutes of blocking reaction. Nucleolin primary antibody (purchased from Santa Cruz Biotechnology, Santa Cruz, Calif.; Catalog No.: sc-8031) diluted 100 times was added into the samples and slowly shaken at 4° C. for 16 hours. After the samples were washed by phosphate buffer solution three times, secondary antibody (purchased from Santa Cruz Biotechnology, Santa Cruz, Calif.; Catalog No.: sc-2005) diluted 1000 times was added for reaction at room temperature for 30 minutes. The slices were stained by diaminobenzidine (DAB), and were counterstained with hematoxylin after washing, followed by dehydration with graded ethanol, and fixation with xylene. The experimental results were photoed with an optical microscope for analysis. Interpretation of the results obtained from the immunohistochemical staining of nucleolin was done by two pathologists independently. Four levels were defined according to the immune response of nucleolin in the nucleus: 0 represented no nuclear staining; 1⁺ represented 1-25% nuclear staining; 2⁺ represented 26-50% nuclear staining; 3⁺ represented >50% nuclear staining. Only 3⁺ was identified as a positive staining result.

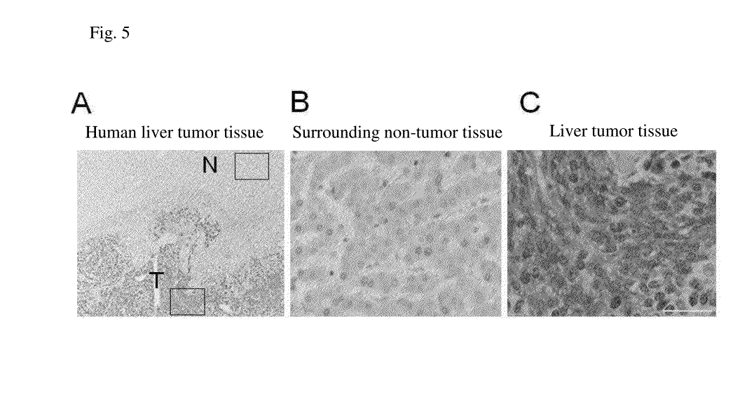

FIG. 5 showed results of immunohistochemical staining for comparing differences between nucleolin protein expression level in human liver tumor and nucleolin protein expression level in non-tumor tissue surrounding the liver tumor. (A): Immunohistochemical staining showing nucleolin protein expression level in liver tumor tissue (T) and surrounding non-tumor tissue (N) of humans (100× magnification). (B): Nucleolin protein expression level in liver non-tumor tissue (N) of humans was presented by capturing and enlarging N region in (A) (400× magnification). (C): Nucleolin protein expression level in liver tumor tissue (T) of humans was presented by capturing and enlarging T region in (A) (400× magnification). Statistical analysis was carried out by using SPSS v17 software to analyze the data. Student's t-test was used to quantitate variables. The two-sided p value less than 0.05 (<0.05) was regarded as significant.

From (A) of FIG. 5, the immunohistochemical staining result showed that nucleolin protein expression level in human liver tumor was significantly higher than that in non-tumor tissue surrounding the liver tumor. The immunohistochemical staining results of human non-tumor tissue and tumor tissue were showed in (B) and (C) of FIG. 5, respectively (400× magnification). Nucleolin showed positive staining in the nucleus, cytoplasm and cell membrane of cancer cells in the tumor tissue (determined by tumor tissue staining results greater than or equal to 50% of the nuclear staining). Traces of nucleolin were found in the non-tumor tissue. Nucleolin expression amount in the liver tumor tissue was significantly higher than that in the non-tumor tissue.

Table 1 showed the correlation between the nucleolin expression level of the above 147 liver cancer patients which had been treated in Kaohsiung Chang Gung Memorial Hospital and the clinicopathological parameters by statistical analysis. The patients were divided into two groups according to immune reaction of nucleolin in the nucleus. Patients with nucleolin staining less than 75% of nuclear staining belonged to the low nucleolin expression group; patients with nucleolin staining greater than or equal to 75% of nuclear staining belonged to the high nucleolin expression group. The statistical results showed that high nucleolin expression level was irrelevant to age, gender, degree of liver cirrhosis, capsule formation, tumor number, and tumor size. However, high nucleolin expression level was highly correlated with tumor grade, vascular invasion, and serum concentration of alpha-fetoprotein, with p values of 0.010, 0.034, and 0.006, respectively, all had significant difference.

TABLE 1
The correlation between the nucleolin expression level of the 147
liver cancer patients and the clinicopathological parameters

| | Low | High | |
|---|---|---|---|
| | nucleolin | nucleolin | |
| | expression | expression | |
| Parameter | (n = 107) | (n = 40) | P value |
| Gender | | | No significant |
| | | | difference (0.59) ^{α} |
| Female (n = 30) | 23 | 7 | |
| Male (n = 117) | 84 | 33 | |
| Liver cirrhosis | | | No significant |
| | | | difference (0.056) ^{α} |
| Yes (n = 88) | 59 | 29 | |
| No (n = 59) | 48 | 11 | |
| Tumor grade | | | *0.010 ^{α} |
| Good differentiation | 32 | 3 | |
| (n = 35) | | | |
| Moderate differentiation | 50 | 21 | |
| (n = 71) | | | |
| Poor differentiation | 25 | 16 | |
| (n = 41) | | | |
| Capsule | | | No significant |
| | | | difference (0.81) ^{α} |
| Yes (n = 82) | 61 | 21 | |
| No (n = 65) | 46 | 19 | |
| Vascular invasion | | | *0.034 ^{α} |
| Yes (n = 74) | 48 | 26 | |
| No (n = 73) | 59 | 14 | |
| Tumor number | | | No significant |
| | | | difference (0.40) ^{α} |
| Single (n = 110) | 82 | 28 | |
| Two or more (n = 37) | 25 | 12 | |
| Age | 53.8 ± | 55.2 ± | No significant |
| | 13.5 | 12.8 | difference (0.59) ^{β} |
| tumor size (cm) | 6.87 ± | 5.65 ± | No significant |
| | 4.21 | 3.14 | difference (0.09) ^{β} |
| serum concentration of | 2712 ± | 3404 ± | *0.006 ^{γ} |
| alpha-fetoprotein (ng/ml) | 7191 | 11329 | |

*Significant differenc (p value < 0.05);
^{α} Chi-square test;
^{β} Student's t -distribution;
^{γ} Mann-Whitney U test

### Example 6

### The Relationship Between Nucleolin Expression Level and Overall Survival of Patients with Liver Cancer

A retrospective analysis of the liver cancer patients which had been treated in Kaohsiung Chang Gung Memorial Hospital from January 1987 to December 1998 was undertaken. This study was approved by Institutional Review Board (IRB) of Kaohsiung Chang Gung Memorial Hospital. Primary tumor samples and corresponding non-tumor tissue were obtained in surgery. Interpretation of the results obtained from the immunohistochemical staining of nucleolin was done by two pathologists independently. Four levels were defined according to the immune response of nucleolin in the nucleus: 0 represented no nuclear staining; 1⁺ represented 1-25% nuclear staining; 2⁺ represented 26-50% nuclear staining; 3⁺ represented >50% nuclear staining. Only 3⁺ was identified as a positive staining result. All patients were Han Chinese. All tissue samples were stained by immunohistochemical staining. All tissue samples were classified as high nucleolin expression group or low nucleolin expression group according to the nucleolin staining results. Kaplan-Meier assay was used to estimate overall survival of patients in the above two groups.

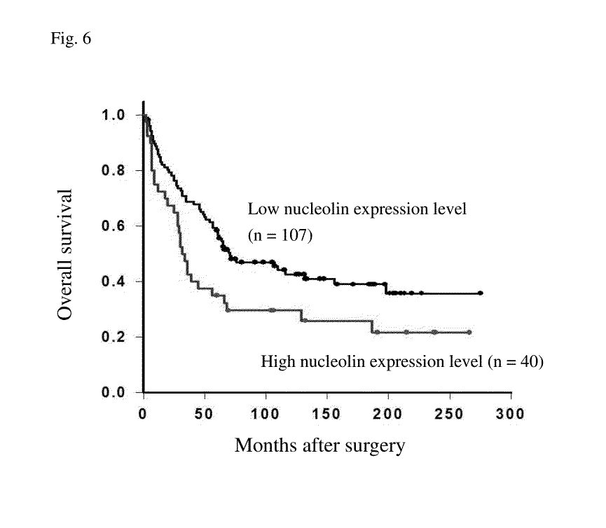

FIG. 6 showed the Kaplan-Meier plot for representing the relationship between nucleolin expression level in human liver tissue samples and overall survival of patients with liver cancer. The perpendicular scale marking displayed survival rate, and the horizontal scale marking displayed months after surgery. The patients were divided into two groups according to immune reaction of nucleolin in the nucleus. Patients with nucleolin staining less than 75% of nuclear staining belonged to the low nucleolin expression group; patients with nucleolin staining greater than or equal to 75% of nuclear staining belonged to the high nucleolin expression group. The black line represented the survival curve of patients with low nucleolin expression in their liver cancer tissue samples (patient number=107); the gray line represented the survival curve of patients with high nucleolin expression in their liver cancer tissue samples (patient number=40). Statistical analysis was carried out by using SPSS v17 software to analyze the data. The Kaplan-Meier assay (log rank test) was used to calculate overall survival. The two-sided p value less than 0.05 (<0.05) was regarded as significant.

FIG. 6 showed the analysis for relationship between nucleolin expression level and overall survival of liver cancer patients in a period of 24 years after surgery. The results showed that 5-year overall survival rate of patients with low nucleolin expression was 58%, and 5-year overall survival rate of patients with high nucleolin expression was 35%. This indicated that 5 yeas after surgery, the overall survival rate of patients with low nucleolin expression level was about 1.7 times of the patients with high nucleolin expression level. The results also showed that 20-year overall survival rate of patients with low nucleolin expression was 36%, and 20-year overall survival rate of patients with high nucleolin expression was 22%. This indicated that 20 yeas after surgery, the overall survival rate of patients with low nucleolin expression level was about 1.6 times of the patients with high nucleolin expression level. The results showed that nucleolin expression could be used to predict prognosis or overall survival of cancer patients after treatment.

### Example 7

### Effects of Nucleolin RNA Interference on Cell Growth and Invasion Ability of Liver Cancer Cells

The 1×10⁴ Huh7 cells were cultured in a 96-well plate for 24 hours. Then the cells were transfected with nucleolin RNA (containing four sets of passenger strands and guide strands with the sequences as follows: 1^{st} passenger strand 5′-CUACGGCUUUCAAUCUCUU-3′ (SEQ ID NO: 4) and 1^{st} guide strand 5′-AAGAGAUUGAAAGCCGUAG-3′ (SEQ ID NO: 5); 2^{nd} passenger strand 5′-UGUUGUGGAUGUCAGAAUU-3′ (SEQ ID NO: 6) and 2^{nd} guide strand 5′-AAUUCUGACAUCCACAACA-3′ (SEQ ID NO: 7); 3^{rd} passenger strand 5′-CCUGUGGUCUCCUUGGAAA-3′ (SEQ ID NO: 8) and 3^{rd} guide strand 5′-UUUCCAAGGAGACCACAGG-3′ (SEQ ID NO: 9); 4^{th} passenger strand 5′-UGAUAGAGCUAACCCUUAU-3′ (SEQ ID NO: 10) and 4^{th} guide strand 5′-AUAAGGGUUAGCUCUAUCA-3′ (SEQ ID NO: 11), all the above passenger strands and guide strands had two overhanging deoxythymidines (tt) at 3′ ends, Catalog No.: sc-29230, purchased from Taiwan, Hong Jin Co., Ltd.) or scrambled RNA (control group, Catalog No.: sc-37007, purchased from Taiwan, Hong Jin Co., Ltd.) for interference of 48 hours. 100 μg of MTT reagent was added into per well during culture. After placed at 37° C. for 4 hours, 500 μl of dimethyl sulfoxide was added into per well to dissolve the formazan precipitate formed. The plate was left to stand for 10 minutes, and then enzyme-linked immunosorbent assay (ELISA) was carried out and the absorbance was read. For cell invasion assay, the lower chamber of the Boyden chamber was placed a culture medium containing 10% serum. The polycarbonate filter at the junction of the upper chamber and the lower chamber (pore size=8 μm) was evenly coated with matrigel. Huh7 cells, interfered Huh7 cells transfected with scrambled RNA, and interfered Huh7 cells transfected with nucleolin RNA (2.5×10⁴) were suspended in 100 ml of serum-free culture medium and placed in the upper chamber of Boyden chamber, respectively. The cells were allowed to attach and stand for 24 hours at 37° C. Cells which had migrated to the bottom of the polycarbonate filter were fixed with methanol and stained by 10% Giemsa. An optical microscope was used to take photographs, the number of cells was counted, and the experimental results were analyzed.

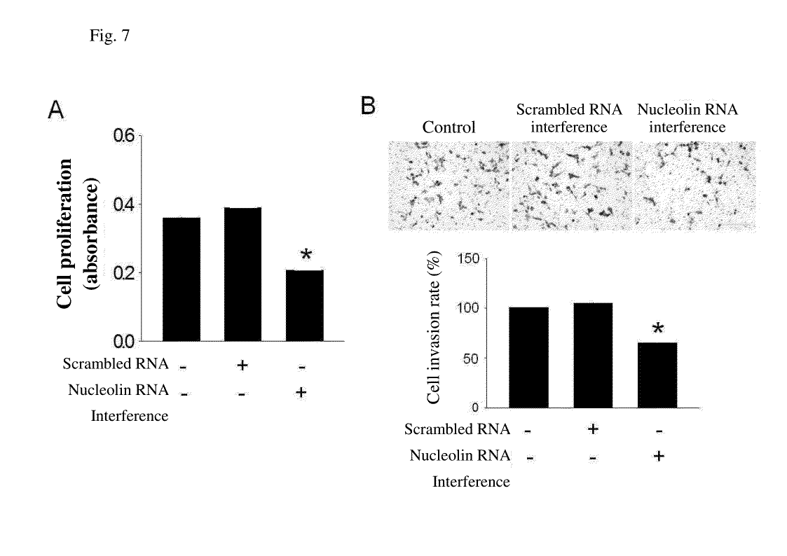

FIG. 7 showed effects of transfecting interfering nucleolin RNA on cell growth and invasive ability of human liver cancer cell line Huh7. (A): The cell proliferation of Huh7 cells, Huh7 cells interfered by transfection of scrambled RNA, and Huh7 cells interfered by transfection of nucleolin RNA was analyzed by cell viability assay. (B): The cell invasion of Huh7 cells, Huh7 cells interfered by transfection of scrambled RNA, and Huh7 cells interfered by transfection of nucleolin RNA was analyzed by cell invasion assay. The upper photos were representative of cell invasion ability. Statistical analysis was carried out by using SPSS v17 software to analyze the data. Student's t-test was used to quantitate variables. The two-sided p value less than 0.05 (<0.05) was regarded as significant. The expressed experimental data was the average of three independent experiments.

From (A) of FIG. 7, the result showed that the growth of liver cancer cells (Huh7) was not affected after interference by transfection of scrambled RNA, but was significantly inhibited after interference by transfection of nucleolin RNA, with the growth rate reduced to about half of the control group. From (B) of FIG. 7, the result showed that the invasion ability of liver cancer cells (Huh7) was not affected after interference by transfection of scrambled RNA, but was significantly inhibited after interference by transfection of nucleolin RNA, with the cell invasion phenomenon reduced to about 60% of the control group.

### Example 8

### Effects of Nucleolin Antibody Neutralization on Cell Growth and Invasion Ability of Liver Cancer Cells

The 1×10⁴ Huh7 cells were cultured in a 96-well plate for 24 hours. Then immunoglobulin G antibody at a concentration of 10 μg/ml (Catalog No.: sc-66931, purchased from Taiwan, Hong Jin Co., Ltd.) or nucleolin antibody at a concentration of 10 μg/ml (Catalog No.: N2662, purchased from Uni-Onward Corp.) was added for antibody neutralization at 37° C. for 24 hours. 100 μg of MTT reagent was added into per well during culture. After placed at 37° C. for 4 hours, 500 μl of dimethyl sulfoxide was added into per well to dissolve the formazan precipitate formed. The plate was left to stand for 10 minutes, and then enzyme-linked immunosorbent assay (ELISA) was carried out and the absorbance was read. For cell invasion assay, the lower chamber of the Boyden chamber was placed a culture medium containing 10% serum. The polycarbonate filter at the junction of the upper chamber and the lower chamber (pore size=8 μm) was evenly coated with matrigel. Huh7 cells were suspended in 100 ml of serum-free culture medium and placed in the upper chamber of Boyden chamber. Immunoglobulin G antibody at a concentration of 10 μg/ml or nucleolin antibody at a concentration of 10 μg/ml was added additionally for antibody neutralization at 37° C. for 24 hours. Cells which had migrated to the bottom of the polycarbonate filter were fixed with methanol and stained by 10% Giemsa. An optical microscope was used to take photographs and the experimental results were analyzed.

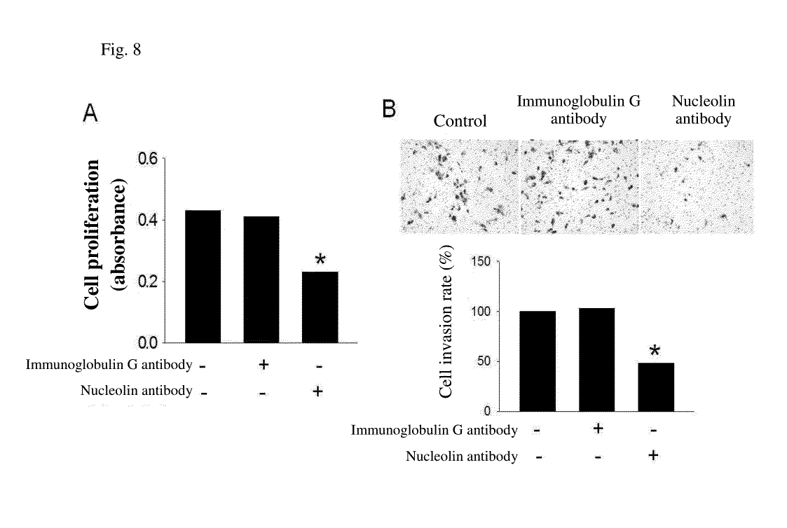

FIG. 8 showed effects of nucleolin antibody neutralization on cell growth and invasive ability of human liver cancer cell line Huh7. (A): The cell proliferation of Huh7 cells, Huh7 cells neutralized by immunoglobulin G antibody, and Huh7 cells neutralized by nucleolin antibody is analyzed by cell viability assay. (B): The cell invasion of Huh7 cells, Huh7 cells neutralized by immunoglobulin G antibody, and Huh7 cells neutralized by nucleolin antibody is analyzed by cell invasion assay. The upper photos are representative of cell invasion ability. Statistical analysis is carried out by using SPSS v17 software to analyze the data. Student's t-test is used to quantitate variables. The two-sided p value less than 0.05 (<0.05) is regarded as significant. The expressed experimental data is the average of three independent experiments.

From (A) of FIG. 8, the result showed that the growth of liver cancer cells (Huh7) was not affected after neutralization by immunoglobulin G antibody (control), but was significantly inhibited after neutralization by nucleolin antibody, with the growth rate reduced to about 60% of the control group. From (B) of FIG. 8, the result showed that the invasion ability of liver cancer cells (Huh7) was not affected after neutralization by immunoglobulin G antibody (control), but was significantly inhibited after neutralization by nucleolin antibody, with the cell invasion phenomenon reduced to about half of the control group.

Immunohistochemical staining and Kaplan-Meier assay are used in the present invention to analyze the condition of liver cancer patients, however, the same result can also be obtained via various other analytical techniques, such as RT-PCR or real-time PCR. Therefore, the scope of the invention should not be limited to analysis results of immunohistochemical staining.

One skilled in the art readily appreciates that the present invention is well adapted to carry out the objects and obtain the ends and advantages mentioned, as well as those inherent therein. The biomarkers of the present invention and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for determining liver cancer, high risk of liver cancer, or high risk of vascular invasion of liver cancer cells in a subject, comprising steps of:
(A) assessing nucleolin expression level in a biological sample obtained from the subject;
(B) comparing the nucleolin expression level with that in a corresponding control sample; and
(C) determining whether the subject has liver cancer, high risk of liver cancer, or high risk of vascular invasion of liver cancer cells in accordance with the result of step (B);
wherein the nucleolin expression level in the biological sample that is higher than that in the corresponding control sample is indicative of the subject having liver cancer, high risk of liver cancer, or high risk of vascular invasion of liver cancer cells.

2. The method of claim 1, wherein the nucleolin expression level is assessed by immunofluorescence staining, western blot, or immunohistochemical staining.

3. The method of claim 1, wherein the biological sample is a liver cell sample, a biopsy sample of liver tissue, a liver homogenate sample, or any combination thereof.

4. The method of claim 1, wherein the corresponding control sample is obtained from a healthy subject, wherein the healthy subject is a subject not suffering from liver cancer.

5. The method of claim 1, which combines with an examination method selected from the group consisting of serum alpha-fetoprotein, ultrasound examination, CT scan (computer tomography) and MRI (magnetic resonance imaging) to facilitate detection of liver cancer and/or determination of cancer tissue invasion or malignancy or vascular invasion.


SEQUENCE LISTING (11 sequences)

SEQ ID NO: 1 - protein, 710 aa, Homo sapiens
  mat_peptide (1)..(710)
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKAAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 2 - DNA, 22 bp, Artificial Sequence
  Forward primer for nucleolin.
atggtgaagctcgcgaaggcag

SEQ ID NO: 3 - DNA, 24 bp, Artificial Sequence
  Reverse primer for nucleolin.
ttcaaacttcgtcttctttccttg

SEQ ID NO: 4 - RNA, 19 nt, Artificial Sequence
  1st siRNA passenger strand.
cuacggcuuucaaucucuu

SEQ ID NO: 5 - RNA, 19 nt, Artificial Sequence
  1st siRNA guide strand.
aagagauugaaagccguag

SEQ ID NO: 6 - RNA, 19 nt, Artificial Sequence
  2nd siRNA passenger strand.
uguuguggaugucagaauu

SEQ ID NO: 7 - RNA, 19 nt, Artificial Sequence
  2nd siRNA guide strand.
aauucugacauccacaaca

SEQ ID NO: 8 - RNA, 19 nt, Artificial Sequence
  3rd siRNA passenger strand.
ccuguggucuccuuggaaa

SEQ ID NO: 9 - RNA, 19 nt, Artificial Sequence
  3rd siRNA guide strand.
uuuccaaggagaccacagg

SEQ ID NO: 10 - RNA, 19 nt, Artificial Sequence
  4rd siRNA passenger strand.
ugauagagcuaacccuuau

SEQ ID NO: 11 - RNA, 19 nt, Artificial Sequence
  4rd siRNA guide strand.
auaaggguuagcucuauca